(19) United States
(12) Patent Application Publication
(10) Pub. No.: US 2012/0178763 A1
(43) Pub. Date: Jul. 12, 2012

(54) Method of Treating Anxious Major Depressive Disorder

(76) Inventors: Joseph Frey, (US); Thomas Hudzik, (US); Mark A. Smith, (US)

(21) Appl. No.: 13/182,822

(22) Filed: Jul. 14, 2011

Related U.S. Application Data
(63) Continuation of application No. 12/468,279, filed on May 19, 2009, now abandoned.
(60) Provisional application No. 61/054,663, filed on May 20, 2008.

(51) Int. Cl. A61K 31/495; A61P 25/22; A61P 25/24

(52) U.S. Cl. 514/255.04

(57) ABSTRACT

The invention is directed to using 4-{(3-aminophenyl)[4-(4-fluorobenzyl)piperazin-1-yl]methyl}-N,N-diethylbenzamide, or enantiomer thereof, or pharmaceutically acceptable salt thereof, and/or mixture thereof to treat anxious major depressive disorder (AMDD).

The invention is directed to using 4-{(3-aminophenyl)[4-(4-fluorobenzyl)piperazin-1-yl]methyl}-N,N-diethylbenzamide, pharmaceutically acceptable salts thereof, or mixtures thereof to treat anxious major depressive disorder (AMDD).

Anxious major depressive disorder (AMDD) is a serious mental illness in which patients suffer from Major depressive disorder (MDD) with prominent comorbid anxiety symptoms. MDD is a common, chronic recurring serious mental illness associated with significant morbidity and often becomes life threatening. Anxiety is a common symptom among patients with MDD. The estimates of prevalence of comorbid anxiety symptoms in patients with MDD have been reported to range from 45-60%. There is increasing evidence that patients with AMDD have greater depressive severity, greater functional impairment, greater risk of depressive relapse, increased risk of suicidality, worse social distress, higher incidence of alcohol and drug abuse, and poorer treatment response and outcome than patients with non-anxious depression. In a recent report by STAR*D, investigators found that remission was significantly less likely to occur in patients with anxious versus nonanxious depression and would likely take longer.

The mu (“μ”), delta (“δ”), and kappa (“κ”) receptors are well established opioid receptors apparent in both the central and peripheral nervous systems of many species including man. Receptor localization studies have shown that δ-opioid receptors reside in areas of the brain implicated in mood regulation. For example, localization of the δ-opioid receptor in the amygdala is consistent with modulation of anxiety states, whereas localization in the cortex and hippocampus is consistent with modulation of depression. The δ-opioid receptor was first identified as a possible target for treating depression and anxiety when heightened anxiety states and depressive-like behaviors were consistently observed in the δ-opioid receptor knockout mouse. A decrease in anxiety has been observed in various animal models when one or more δ-opioid receptors was activated. Additionally, a number of investigators have found selective δ-opioid receptor agonists have antidepressant-like properties in models such as the forced swim test.

While there are medicines available to treat depression, a large percentage of patients with AMDD fail to respond to treatment with currently available antidepressants. The primary biochemical effects of current antidepressants are exerted by increasing the intrasynaptic levels of monoamines, and as such, have had limited efficacy in treating AMDD. The above data highlight the importance of developing medications that not only effectively treat depression but also effectively treat anxiety. As a result of the limited efficacy of currently available AMDD treatments, efforts have been undertaken to develop δ-opioid receptor ligands that are therapeutically effective in treating AMDD. More specifically, efforts have been focused on developing selective δ-opioid receptor ligands. Selective δ-opioid receptor ligands advantageously cause less side effects than non-selective δ-opioid receptor ligands.

Described herein is a method for treating anxious major depressive disorder in a warm-blooded animal, comprising administering to said animal in need of such treatment a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof or mixture thereof.

Further described herein is a method for treating anxious major depressive disorder in a warm-blooded animal, comprising administering to said animal in need of such treatment a pharmaceutical composition comprising a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, and a pharmaceutically acceptable carrier.

Still further described herein is use of 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof in the manufacture of a medicament for the therapy of anxious major depressive disorder.

Even further described herein is a pharmaceutical composition comprising a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, and a pharmaceutically acceptable carrier for the treatment of anxious major depressive disorder.

Yet even further described herein is a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof for the treatment of anxious major depressive disorder.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Unless specifically stated otherwise herein, references made in the singular may also include the plural. For example, “a” and “an” may refer to either one, or one or more. Embodiments identified herein as exemplary are intended to be illustrative and not limiting. The definitions set forth herein take precedence over definitions set forth in any patent, patent application, and/or patent application publication that may be incorporated herein by reference.

“Enantiomerically pure” refers to a compound containing at least 75% of the named enantiomer out of the total amount of the two possible enantiomers contained therein. In another embodiment, “enantiomerically pure” refers to a compound containing at least 90% of the named enantiomer out of the total amount of the two possible enantiomers contained therein. In a further embodiment, “enantiomerically pure” refers to a compound containing at least 95% of the named enantiomer out the total amount of the two possible enantiomers contained therein.

The term “pharmaceutically-acceptable”, as employed herein, indicates the subject matter being identified as “pharmaceutically acceptable” is suitable and physiologically acceptable for administration to a patient. For example, the term “pharmaceutically acceptable salt(s)” denotes suitable and physiologically acceptable salt(s).

The term “therapy” also includes “prophylaxis” unless there are specific indications to the contrary. The term “therapy” encompasses mitigating a pre-existing disease state, acute or chronic, as well as a recurring condition. The term “therapeutic” and “therapeutically” should be construed in accordance with this definition, which also encompasses prophylactic therapies for prevention of recurring conditions and continued therapy for chronic disorders.

The phrase “4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof” refers to the free base of 4-{(3-aminophenyl)[4-(4-fluorobenzyl)piperazin-1-yl]methyl}-N,N-diethylbenzamide, enantiomer(s) of the free base of 4-{(3-aminophenyl)[4-(4-fluorobenzyl)piperazin-1-yl]methyl}-N,N-diethylbenzamide, pharmaceutically acceptable salt(s) of the free base of 4-{(3-aminophenyl)[4-(4-fluorobenzyl)piperazin-1-yl]methyl}-N,N-diethylbenzamide, pharmaceutically acceptable salt(s) of the enantiomer(s) of the free base of 4-{(3-aminophenyl)[4-(4-fluorobenzyl)piperazin-1-yl]methyl}-N,N-diethylbenzamide, and/or mixture(s) of any of the foregoing.

The phrase “4-{(R)-(3-aminophenyl)[4-(4-fluorobenzyl)piperazin-1-yl]methyl}-N,N-diethylbenzamide, pharmaceutically acceptable salt thereof, or mixture thereof” refers to the free base of 4-{(R)-(3-aminophenyl)[4-(4-fluorobenzyl)piperazin-1-yl]methyl}-N,N-diethylbenzamide, pharmaceutically acceptable salt(s) of the free base of 4-{(R)-(3-aminophenyl)[4-(4-fluorobenzyl)piperazin-1-yl]methyl}-N,N-diethylbenzamide, and/or mixtures of any of the foregoing.

The term “therapeutically-effective amount” refers to that amount of a compound sufficient to modulate one or more of the symptoms of the condition or disease being treated. A “therapeutically effective amount” and/or dosage range for compound used in the method of treatment of the invention may be determined by one of ordinary skill in the art via known criteria including age, weight, and response of the individual patient, and interpreted within the context of the disease being treated and/or prevented. Exemplary single or divided dosage amounts for a mammal may be from about 0.01 to about 300 mg/kg/day.

One embodiment is directed to a method for treating anxious major depressive disorder in a warm-blooded animal, comprising administering to said animal in need of such treatment a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof.

Another embodiment is directed to a method for treating anxious major depressive disorder in a warm-blooded animal, comprising administering to said animal in need of such treatment a therapeutically effective amount of a compound comprising 4-{(R)-(3-aminophenyl)[4-(4-fluorobenzyl)piperazin-1-yl]methyl}-N,N-diethylbenzamide, or pharmaceutically acceptable salt thereof, or mixture thereof.

A further embodiment is directed to a method for treating anxious major depressive disorder in a warm-blooded animal, comprising administering to said animal in need of such treatment a therapeutically effective amount of a compound comprising 4-{(R)-(3-aminophenyl)[4-(4-fluorobenzyl)piperazin-1-yl]methyl}-N,N-diethylbenzamide sesquifumarate.

Yet another embodiment is directed to a method for treating anxious major depressive disorder in a warm-blooded animal, comprising administering to said animal in need of such treatment a therapeutically effective amount of an enantiomerically pure compound comprising at least 75% 4-{(R)-(3-aminophenyl)[4-(4-fluorobenzyl)piperazin-1-yl]methyl}-N,N-diethylbenzamide, or pharmaceutically acceptable salt thereof, or mixture thereof.

Yet still another embodiment is directed to a method for treating anxious major depressive disorder in a warm-blooded animal, comprising administering to said animal in need of such treatment a therapeutically effective amount of an enantiomerically pure compound comprising at least 90% 4-{(R)-(3-aminophenyl)[4-(4-fluorobenzyl)piperazin-1-yl]methyl}-N,N-diethylbenzamide, or pharmaceutically acceptable salt thereof, or mixture thereof.

A further embodiment is directed to a method for treating anxious major depressive disorder in a warm-blooded animal, comprising administering to said animal in need of such treatment a therapeutically effective amount of an enantiomerically pure compound comprising at least 95% 4-{(R)-(3-aminophenyl)[4-(4-fluorobenzyl)piperazin-1-yl]methyl}-N,N-diethylbenzamide, or pharmaceutically acceptable salt thereof, or mixture thereof.

Another embodiment is directed to a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof for the treatment of anxious major depressive disorder.

Yet another embodiment is directed to a compound comprising 4-{(R)-(3-aminophenyl)[4-(4-fluorobenzyl)piperazin-1-yl]methyl}-N,N-diethylbenzamide, or pharmaceutically acceptable salt thereof, or mixture thereof for the treatment of anxious major depressive disorder.

Yet even still another embodiment is directed to a compound comprising 4-{(R)-(3-aminophenyl)[4-(4-fluorobenzyl)piperazin-1-yl]methyl}-N,N-diethylbenzamide sesquifumarate for the treatment of anxious major depressive disorder.

Still yet another embodiment is directed to a compound comprising an enantiomerically pure compound comprising at least 75% 4-{(R)-(3-aminophenyl)[4-(4-fluorobenzyl)piperazin-1-yl]methyl}-N,N-diethylbenzamide, or pharmaceutically acceptable salt thereof, or mixture thereof for the treatment of anxious major depressive disorder.

A still yet further embodiment is directed to a compound comprising an enantiomerically pure compound comprising at least 90% 4-{(R)-(3-aminophenyl)[4-(4-fluorobenzyl)piperazin-1-yl]methyl}-N,N-diethylbenzamide, or pharmaceutically acceptable salt thereof, or mixture thereof for the treatment of anxious major depressive disorder.

An even further embodiment is directed to a compound comprising an enantiomerically pure compound comprising at least 95% 4-{(R)-(3-aminophenyl)[4-(4-fluorobenzyl)piperazin-1-yl]methyl}-N,N-diethylbenzamide, or pharmaceutically acceptable salt thereof, or mixture thereof for the treatment of anxious major depressive disorder.

A still further embodiment is directed to a method for treating anxious major depressive disorder in a warm-blooded animal, comprising administering to said animal in need of such treatment a pharmaceutical composition comprising a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, and a pharmaceutically acceptable carrier.

Yet still a further embodiment is directed to a method for treating anxious major depressive disorder in a warm-blooded animal, comprising administering to said animal in need of such treatment a pharmaceutical composition comprising a therapeutically effective amount of a compound comprising 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier.

A yet further embodiment is directed to a method for treating anxious major depressive disorder in a warm-blooded animal, comprising administering to said animal in need of such treatment a pharmaceutical composition comprising a therapeutically effective amount of a compound comprising 4-{(R)-(3-aminophenyl)[4-(4-fluorobenzyl)piperazin-1-yl]methyl}-N,N-diethylbenzamide sesquifumarate and a pharmaceutically acceptable carrier.

An even further embodiment is directed to a method for treating anxious major depressive disorder in a warm-blooded animal, comprising administering to said animal in need of such treatment a pharmaceutical composition comprising a therapeutically effective amount of an enantiomerically pure compound comprising at least 75% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier.

A still even further embodiment is directed to a method for treating anxious major depressive disorder in a warm-blooded animal, comprising administering to said animal in need of such treatment a pharmaceutical composition comprising a therapeutically effective amount of an enantiomerically pure compound comprising at least 90% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier.

A yet still even further embodiment is directed to a method for treating anxious major depressive disorder in a warm-blooded animal, comprising administering to said animal in need of such treatment a pharmaceutical composition comprising a therapeutically effective amount of an enantiomerically pure compound comprising at least 95% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier.

Still yet a further embodiment is directed to a pharmaceutical composition comprising a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, and a pharmaceutically acceptable carrier for the treatment of anxious major depressive disorder.

Even still yet a further embodiment is directed to a pharmaceutical composition comprising a therapeutically effective amount of a compound comprising 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier for the treatment of anxious major depressive disorder.

A further embodiment is directed to a pharmaceutical composition comprising a therapeutically effective amount of a compound comprising 4-{(R)-(3-aminophenyl)[4-(4-fluorobenzyl)piperazin-1-yl]methyl}-N,N-diethylbenzamide sesquifumarate, and a pharmaceutically acceptable carrier for the treatment of anxious major depressive disorder.

A still further embodiment is directed to a pharmaceutical composition comprising a therapeutically effective amount of a compound comprising an enantiomerically pure compound comprising 75% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier for the treatment of anxious major depressive disorder.

A yet still further embodiment is directed to a pharmaceutical composition comprising a therapeutically effective amount of a compound comprising an enantiomerically pure compound comprising 90% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier for the treatment of anxious major depressive disorder.

A yet further embodiment is directed to a pharmaceutical composition comprising a therapeutically effective amount of a compound comprising an enantiomerically pure compound comprising 95% 4-{(R)-(3-aminophenyl)[4-(4-fluorobenzyl)piperazin-1-yl]methyl}-N,N-diethylbenzamide, or pharmaceutically acceptable salt thereof, or mixture thereof, and a pharmaceutically acceptable carrier for the treatment of anxious major depressive disorder.

In one embodiment, the warm-blooded animal is a mammalian species. Exemplary mammalian species include but are not limited to, for example, humans and domestic animals, such as, for example, dogs, cats, and horses.

In a further embodiment, the warm-blooded animal is a human.

In one embodiment, the pharmaceutically acceptable carrier is selected from a solid carrier and a liquid carrier.

Solid carriers include, but are not limited to, for example, powders, tablets, dispersible granules, capsules, cachets, and suppositories. A solid carrier can be one or more substances, which may also act as diluents, flavoring agents, solubilizers, lubricants, suspending agents, binders, or table disintegrating agents. A solid carrier can also be an encapsulating material.

In powders, the carrier is a finely divided solid mixed with a finely divided compound of the invention.

In tablets, the compound of the invention is mixed with a carrier having the necessary binding properties and in proportions suitable to be compacted into the shape and size desired.

In a suppository, a low-melting wax, such as, for example, a mixture of fatty acid glycerides and cocoa butter is first melted and a compound of the invention is dispersed therein by, for example, stirring. The molten homogeneous mixture in then poured into convenient size molds and allowed to cool and solidify.

Suitable carriers, include but are not limited to, for example, magnesium carbonate, magnesium stearate, talc, lactose, sugar, pectin, dextrin, starch, tragacanth, methyl cellulose, sodium carboxymethyl cellulose, a low-melting wax, and cocoa butter.

The term “composition” is also intended to include the formulation of a compound of the invention with encapsulating material as a carrier to provide a capsule in which a compound of the invention (with or without other carriers) is surrounded by a carrier which is thus in association with it. Similarly, cachets are included.

Tablets, powders, cachets, and capsules can be used as solid dosage forms suitable for oral administration.

Liquid dosage forms include, but are not limited to, for example, solutions, suspensions, and emulsions. For example, sterile water or propylene glycol solutions of a compound of the invention may be liquid preparations suitable for parenteral administration. Liquid dosage forms can also be formulated as an aqueous polyethylene glycol solution.

Liquid dosage forms for oral administration can be prepared by dissolving a compound of the invention in water and adding suitable colorants, flavoring agents, stabilizers, and thickening agents as desired. Liquid suspensions for oral administration can be made by dispersing a finely divided compound of the invention in water together with a suspending agent, such as, for example, natural synthetic gums, resins, methyl cellulose, and sodium carboxymethyl cellulose.

One embodiment is directed to a pharmaceutical composition comprising from 0.05% to 99% w (percent by weight) of at least one compound of the invention, all percentages by weight being based on total composition.

Another embodiment is directed to a pharmaceutical composition comprising from 0.10 to 50% w (percent by weight) of at least one compound of the invention, all percentages by weight being based on total composition.

Certain compounds of the invention may exist in solvated, for example hydrated, as well as unsolvated forms. As a result, the invention encompasses methods of treatment with solvated forms of the compound.

Pharmaceutically acceptable salts of a compound of the invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the pharmaceutically acceptable salt is an acid addition salt such as a hydrochloride, hydrobromide, phosphate, acetate, fumarate, maleate, tartrate, citrate, methanesulphonate or p-toluenesulphonate.

In yet another embodiment, the pharmaceutically acceptable salt is a sesquifumarate or mono-HCL salt.

Another embodiment is directed to the use of 4-{(3-aminophenyl)[4-(4-fluorobenzyl)piperazin-1-yl]methyl}-N,N-diethylbenzamide or enantiomer thereof, or pharmaceutically acceptable salt thereof; or mixture thereof for the manufacture of a medicament for the therapy of anxious major depressive disorder.

Yet another embodiment is directed to the use of 4-{(R)-(3-aminophenyl)[4-(4-fluorobenzyl)piperazin-1-yl]methyl}-N,N-diethylbenzamide, pharmaceutically acceptable salt thereof, or mixture thereof for the manufacture of a medicament for the therapy of anxious major depressive disorder.

Still yet another embodiment is directed to the use of an enantiomerically pure compound comprising at least 75% 4-{(R)-(3-aminophenyl)[4-(4-fluorobenzyl)piperazin-1-yl]methyl}-N,N-diethylbenzamide, or pharmaceutically acceptable salt thereof, or mixture thereof for the manufacture of a medicament for the therapy of anxious major depressive disorder.

An even further embodiment is directed to the use of an enantiomerically pure compound comprising at least 90% 4-{(R)-(3-aminophenyl)[4-(4-fluorobenzyl)piperazin-1-yl]methyl}-N,N-diethylbenzamide, or pharmaceutically acceptable salt thereof, or mixture thereof for the manufacture of a medicament for the therapy of anxious major depressive disorder.

A still further embodiment is directed to the use of an enantiomerically pure compound comprising at least 95% 4-{(R)-(3-aminophenyl)[4-(4-fluorobenzyl)piperazin-1-yl]methyl}-N,N-diethylbenzamide, or pharmaceutically acceptable salt thereof, or mixture thereof for the manufacture of a medicament for the therapy of anxious major depressive disorder.

Yet another embodiment is directed to a method for treating a patient suffering from anxious major depressive disorder with a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, or a pharmaceutical composition or formulation comprising a therapeutically effective amount of a compound comprising 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof, and at least one other pharmaceutically active compound, wherein said compound and other pharmaceutically active compound are administered concurrently, simultaneously, sequentially or separately and said at least one other pharmaceutically active compound is selected from the following:

(i) antidepressants, such as, for example, agomelatine, amitriptyline, amoxapine, bupropion, citalopram, clomipramine, desipramine, doxepin, duloxetine, escitalopram, fluvoxamine, fluoxetine, gepirone, imipramine, ipsapirone, isocarboxazid, maprotiline, mirtazepine, nortriptyline, nefazodone, paroxetine, phenelzine, protriptyline, ramelteon, reboxetine, robalzotan, selegiline, sertraline, sibutramine, thionisoxetine, tranylcypromaine, trazodone, trimipramine, venlafaxine, and equivalents and pharmaceutically active isomer(s) and metabolite(s) thereof;

(ii) antipsychotics, such as, for example, amisulpride, aripiprazole, asenapine, benzisoxidil, bifeprunox, carbamazepine, clozapine, chlorpromazine, debenzapines, dibenzapine, divalproex, droperidol, fluphenazine, haloperidol, iloperidone, loxapine, mesoridazine, molindone, olanzapine, paliperidone, perphenazine, phenothiazine, phenylbutylpiperidine, pimozide, prochlorperazine, quetiapine, risperidone, sertindole, sulpiride, suproclone, thioridazine, thiothixene, trifluoperazine, trimetozine, valproate, valproic acid, zotepine, ziprasidone, and equivalents and pharmaceutically active isomer(s) and metabolite(s) thereof;

(iii) anxiolytics, such as, for example, alnespirone, azapirones, benzodiazepines, and barbiturates, such as, for example, adinazolam, alprazolam, balezepam, bentazepam, bromazepam, brotizolam, buspirone, clonazepam, clorazepate, chlordiazepoxide, cyprazepam, diazepam, estazolam, fenobam, flunitrazepam, flurazepam, fosazepam, lorazepam, lormetazepam, meprobamate, midazolam, nitrazepam, oxazepam, prazepam, quazepam, reclazepam, suriclone, tracazolate, trepipam, temazepam, triazolam, uldazepam, zolazepam, and equivalents and pharmaceutically active isomer(s) and metabolite(s) thereof;

(iv) anticonvulsants, such as, for example, carbamazepine, oxcarbazepine, valproate, lamotrogine, gabapentin, topiramate, phenyloin, ethosuximide, and equivalents and pharmaceutically active isomer(s) and metabolite(s) thereof;

(v) Alzheimer's therapies, such as, for example, donepezil, galantamine, memantine, rivastigmine, tacrine, and equivalents and pharmaceutically active isomer(s) and metabolite(s) thereof;

(vi) Parkinson's therapies and agents for the treatment of extrapyramidal symtpoms, such as, for example, levodopa, carbidopa, amantadine, pramipexole, ropinirole, pergolide, cabergoline, apomorphine, bromocriptine, MAOB inhibitors (i.e. selegine and rasagiline), COMT inhibitors (i.e. entacapone and tolcapone), alpha-2 inhibitors, anticholinergics (i.e., benztropine, biperiden, orphenadrine, procyclidine, and trihexyphenidyl), dopamine reuptake inhibitors, NMDA antagonists, Nicotine agonists, Dopamine agonists, and inhibitors of neuronal nitric oxide synthase, and equivalents and pharmaceutically active isomer(s) and metabolite(s) thereof;

(vii) migraine therapies, such as, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(viii) stroke therapies, such as, for example, abciximab, activase, NXY-059, citicoline, crobenetine, desmoteplase, repinotan, traxoprodil, and equivalents and pharmaceutically active isomer(s) and metabolite(s) thereof;

(ix) urinary incontinence therapies, such as, for example, darafenacin, dicyclomine, falvoxate, imipramine, desipramine, oxybutynin, propiverine, propanthedine, robalzotan, solifenacin, alfazosin, doxazosin, terazosin, tolterodine, and equivalents and pharmaceutically active isomer(s) and metabolite(s) thereof;

(x) neuropathic pain therapies, such as, for example, gabapentin, lidoderm, pregablin, and equivalents and pharmaceutically active isomer(s) and metabolite(s) thereof;

(xi) nociceptive pain therapies, such as, for example, celecoxib, codeine, diclofenac, etoricoxib, fentanyl, hydrocodone, hydromorphone, levo-alpha-acetylmethadol, loxoprofen, lumiracoxib, meperidine, methadone, morphine, naproxen, oxycodone, paracetamol, propoxyphene, rofecoxib, sufentanyl, valdecoxib, and equivalents and pharmaceutically active isomer(s) and metabolite(s) thereof;

(xii) insomnia therapies and sedative hypnotics, such as, for example, agomelatine, allobarbital, alonimid, amobarbital, benzoctamine, butabarbital, capuride, chloral hydrate, clonazepam, chlorazepate, cloperidone, clorethate, dexclamol, estazolam, eszopiclone, ethchlorvynol, etomidate, flurazepam, glutethimide, halazepam, hydroxyzine, mecloqualone, melatonin, mephobarbital, methaqualone, midaflur, midazolam, nisobamate, pagoclone, pentobarbital, perlapine, phenobarbital, propofol, quazepam, ramelteon, roletamide, suproclone, temazepam, triazolam, triclofos, secobarbital, zaleplon, zolpidem, zopiclone and equivalents and pharmaceutically active isomer(s) and metabolite(s) thereof;

(xiii) mood stabilizers, such as, for example, carbamazepine, divalproex, gabapentin, lamotrigine, lithium, olanzapine, oxycarbazepine, quetiapine, vaiproate, valproic acid, verapamil, and equivalents and pharmaceutically active isomer(s) and metabolite(s) thereof;

(xiv) medications for treating obesity, such as, for example, orlistat, sibutramine, rimonabant, and equivalents and pharmaceutically active isomer(s) and metabolite(s) thereof;

(xv) agents for treating ADHD, such as, for example, amphetamine, methamphetamine, dextroamphetamine, atomoxetine, methylphenidate, dexmethylphenidate, modafinil, and equivalents and pharmaceutically active isomer(s) and metabolite(s) thereof; and

(xvi) agents used to treat substance abuse disorders, dependence, and withdrawal, such as, for example, nicotine replacement therapies (i.e., gum, patches, and nasal spray); nicotinergic receptor agonists, partial agonists, and antagonists, (e.g. varenicline); acomprosate, bupropion, clonidine, disulfuram, methadone, naloxone, naltrexone, and equivalents and pharmaceutically active isomer(s) and metabolite(s) thereof.

Such combination treatment employs 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof within the dosage range described herein and the other pharmaceutically active compound or compounds within approved dosage ranges and/or the dosage described in relevant publications.

When a human suffering from anxious major depressive disorder is treated, 4-{(3-aminophenyl)[4-(4-fluorobenzyl)piperazin-1-yl]methyl}-N,N-diethylbenzamide or enantiomer thereof, or pharmaceutically acceptable salt thereof, or mixture thereof may be administered in the form of a conventional pharmaceutical composition by any route including, but not limited to, orally, intramuscularly, subcutaneously, topically, intranasally, intraperitoneally, intrathoracially, intravenously, epidurally, intrathecally, intracerebroventricularly, and injection into the joints.

In one embodiment of the invention, the route of administration is selected from oral, intravenous, and intramuscular.

The specific dose level and frequency of dosage for any particular subject may vary and generally depends on a variety of factors, including, but not limited to, for example, the bioavailability of the compounds of the present invention in the administered form; metabolic stability and length of action of the specific compounds of the present invention; species, age, body weight, general health, sex, and diet of the subject; mode and time of administration; rate of excretion; drug combination; and severity of the particular condition; and any other factors normally considered by an attending physician when determining the individual regimen and dosage level that is the most appropriate for a particular patient.

In general, a compound of the invention can be prepared in accordance with the following schemes and general knowledge of one skilled in the art and/or in accordance with the methods set forth in the example(s) that follow. Solvents, temperatures, pressures, and other reaction conditions may readily be selected by one of ordinary skill in the art. Starting materials are commercially available or readily prepared by one skilled in the art. Combinatorial techniques can be employed in the preparation of compounds, for example, where the intermediates possess groups suitable for these techniques.

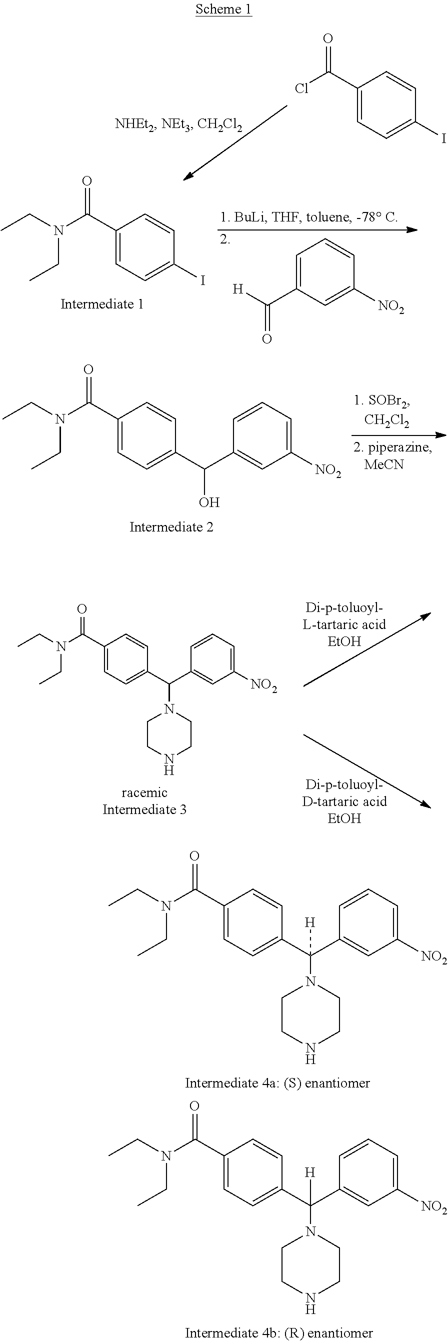

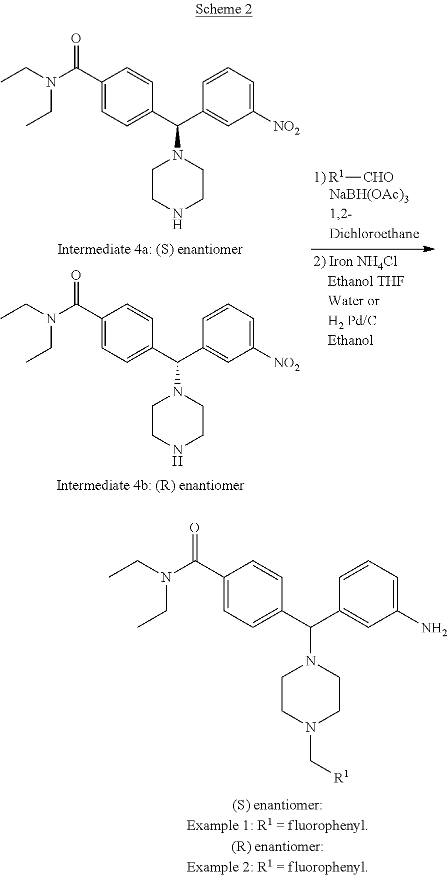

### Biological Evaluation

At least one compound of the invention including the compounds described in the examples hereof, when tested in at least one in vitro assay described below is active towards δ receptors. Particularly, at least one compound of the invention is an effective δ receptor ligand. The in vitro activity may be related to in vivo activity but may not be linearly correlated with binding affinity. In at least one in vitro assay described herein, at least one compound of the invention is tested for activity toward δ receptors and IC₅₀ is obtained to determine the selective activity for a particular compound towards δ receptors. In the current context, IC₅₀ generally refers to the concentration of the compound at which 50% displacement of a standard radioactive δ receptor ligand has been observed.

The activity of at least one compound of the invention towards κ and μ receptors can also be measured in a similar assay.

### In Vitro Models

### Cell Culture

Human 293S cells expressing cloned human κ, δ and μ receptors and neomycin resistance are grown in suspension at 37° C. and 5% CO₂ in shaker flasks containing calcium-free DMEM10% FBS, 5% BCS, 0.1% Pluronic F-68, and 600 μg/ml geneticin.

Rat brains are weighed and rinsed in ice-cold PBS (containing 2.5 mM EDTA, pH 7.4). The brains are homogenized with a polytron for 30 sec (rat) in ice-cold lysis buffer (50 mM Tris, pH 7.0, 2.5 mM EDTA, with phenylmethylsulfonyl fluoride added just prior use to 0.5 MmM from a 0.5M stock in DMSO:ethanol).

### Membrane Preparation

Cells are pelleted and resuspended in lysis buffer (50 mM Tris, pH 7.0, 2.5 mM EDTA, with PMSF added just prior to use to 0.1 mM from a 0.1 M stock in ethanol), incubated on ice for 15 min, then homogenized with a polytron for 30 sec. The suspension is spun at 1000 g (max) for 10 min at 4° C. The supernatant is saved on ice and the pellets resuspended and spun as before. The supernatants from both spins are combined and spun at 46,000 g (max) for 30 min. The pellets are resuspended in cold Tris buffer (50 mM Tris/Cl, pH 7.0) and spun again. The final pellets are resuspended in membrane buffer (50 mM Tris, 0.32 M sucrose, pH 7.0). Aliquots (1 ml) in polypropylene tubes are frozen in dry ice/ethanol and stored at −70° C. until use. The protein concentrations are determined by a modified Lowry assay with sodium dodecyl sulfate.

### Binding Assays

Membranes are thawed at 37° C., cooled on ice, (or kept on ice if not used immediately) passed 3 times through a 25-gauge needle, and diluted into binding buffer (50 mM Tris, 3 mM MgCl₂, 1 mg/ml BSA (Sigma A-7888), pH 7.4, which is stored at 4° C. after filtration through a 0.22 m filter, and to which has been freshly added 5 μg/ml aprotinin, 10 μM bestatin, 10 μM diprotin A if the membranes are derived from tissue (rat, mouse, monkey, no DTT). Aliquots of 100 μl are added to iced 12×75 mm polypropylene tubes containing 100 μl of the appropriate radioligand and 100 μl of test compound at various concentrations. Total (TB) and nonspecific (NS) binding are determined in the absence and presence of 10 μM naloxone respectively. The tubes are vortexed and incubated at 25° C. for 60-75 min, after which time the contents are rapidly vacuum-filtered and washed with about 12 ml/tube iced wash buffer (50 mM Tris, pH 7.0, 3 mM MgCl₂) through GF/B filters (Whatman) presoaked for at least 2 h in 0.1% polyethyleneimine. The radioactivity (dpm) retained on the filters is measured with a beta counter after soaking the filters for at least 12 h in minivials containing 6-7 ml scintillation fluid. If the assay is set up in 96-place deep well plates, the filtration is over 96-place PEI-soaked unifilters, which are washed with 3×1 ml wash buffer, and dried in an oven at 55° C. for 2 h. The filter plates are counted in a TopCount (Packard) after adding 50 μl MS-20 scintillation fluid/well. In the case of assays performed in 96 deep well plates, the IC₅₀ of the compounds are evaluated from 10-point displacement curves in the case of Delta, and 5-point displacement curves in the case of Mu and Kappa. The assay is done in 300 μl with the appropriate amount of membrane protein (2 μg, 35 μg, and 1 μg, in the case of Delta, Mu, and Kappa, respectively) and 50000-80000 dpm/well of the appropriate tracer (125I-Deltorphin II, 125I-FK33824, and 125I-DPDYN for Delta, Mu, and Kappa, respectively). The total binding and non-specific binding are determined in absence and presence of 10 μM of Naloxone.

### Functional Assays

The agonist activity of at least one compound of the invention can be measured by determining the degree to which the compound receptor complex activates the binding of GTP to G-proteins to which the receptors are coupled. In the GTP binding assay, GTP[γ]³⁵S is combined with test compounds and membranes from HEK-293S cells expressing the cloned human opioid receptors or from homogenised rat or mouse brain. Agonists stimulate GTP[γ]³⁵S binding in these membranes. The EC₅₀ and Eₘₐₓ values of the compounds are determined from dose-response curves. Right shifts of the dose response curve by the delta antagonist naltrindole are performed to verify that agonist activity is mediated through delta receptors. For human δ receptor functional assays, EC₅₀ (low) is measured when the human δ receptors used in the assay were expressed at lower levels in comparison with those used in determining EC₅₀ (high). The Eₘₐₓ values were determined in relation to the standard δ agonist SNC80, i.e., higher than 100% is a compound that has better efficacy than SNC80.

### Procedure for Rat Brain GTP

Rat brain membranes are thawed at 37° C., passed 3 times through a 25-gauge blunt-end needle and diluted in the GTPγS binding (50 mM Hepes, 20 mM NaOH, 100 mM NaCl, 1 mM EDTA, 5 mM MgCl₂, pH 7.4, Add fresh: 1 mM DTT, 0.1% BSA). 120 μM GDP final is added membranes dilutions. The EC₅₀ and Emax of compounds are evaluated from 10-point dose-response curves done in 300 μl with the appropriate amount of membrane protein (20 μg/well) and 100000-130000 dpm of GTPγ³⁵S per well (0.11-0.14 nM). The basal and maximal stimulated binding are determined in absence and presence of 3 μM SNC80. The assay performed on HEK 293S cells stably expressing cloned Delta receptors is done in a slightly different buffer (50 mM Hepes, 20 mM NaOH, 200 mM NaCl, 1 mM EDTA, 5 mM MgCl₂, pH 7.4, Add fresh: 0.5% BSA, no DTT) and with a 3 μM final conc. of GDP.

### Data Analysis

The specific binding (SB) was calculated as TB-NS, and the SB in the presence of various test compounds was expressed as percentage of control SB. Values of IC₅₀ and Hill coefficient (n_{H}) for ligands in displacing specifically bound radioligand were calculated from logit plots or curve fitting programs such as Ligand, GraphPad Prism, SigmaPlot, or ReceptorFit. Values of Kᵢ were calculated from the Cheng-Prussoff equation. Mean±S.E.M. values of IC₅₀, Kᵢ and n_{H} were reported for ligands tested in at least three displacement curves.

Set forth in Table 1 hereinbelow are IC₅₀ values generated in accordance with the binding and/or functional assays as essentially described hereinabove.

TABLE 1

| | | | | | EC50 hδ | | EC50 hδ | | |
|---|---|---|---|---|---|---|---|---|---|
| | | | | EC50 hδ | (low) | EC50 hδ | (high) | | EC50 rb |
| Structure | IC50 hd | IC50 hk | IC50 hm | (low) | Emax | (high) | Emax | EC50 rb | Emax |
| 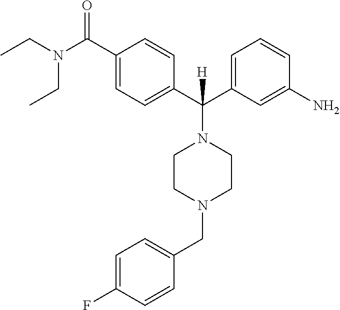 | 0.587 | 5524 | 715 | 20.63 | 95.3 | 4.18 | 103.8 | 22.82 | 130.3 |
| (Example 2) | | | | | | | | | |
| 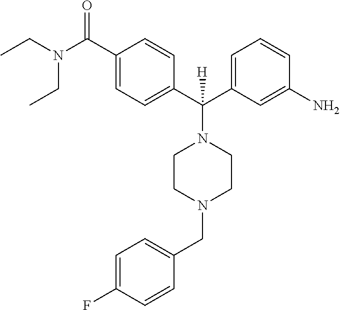 | 8.80 | >10000 | 3316 | N/A | N/A | 100.3 | 89.09 | 465.9 | 66.32 |
| (Example 1) | | | | | | | | | |

N/A: not available

### Receptor Saturation Experiments

Radioligand Kd values are determined by performing the binding assays on cell membranes with the appropriate radioligands at concentrations ranging from 0.2 to 5 times the estimated Kd (up to 10 times if amounts of radioligand required are feasible). The specific radioligand binding is expressed as pmole/mg membrane protein. Values of K_{δ} and Bₘₐₓ from individual experiments are obtained from nonlinear fits of specifically bound (B) vs. nM free (F) radioligand from individual according to a one-site model.

### EXAMPLES

The invention will further be described in more detail by the following Examples which describe methods whereby compounds of the present invention may be prepared, purified, analyzed and biologically tested, and which are not to be construed as limiting the invention.

Unless specified otherwise, the compounds discussed herein were generally named by following the examples and rules stated in Nomenclature of Organic Chemistry, Sections A, B, C, D, E, F, and H, Pergamon Press, Oxford, 1979. Optionally, names of the compounds discussed herein may be generated using a chemical naming program, such as, for example, ACD/ChemSketch, Version 5.09/September 2001, Advanced Chemistry Development, Inc., Toronto, Canada.

Chiral purity was determined by HPLC using the following conditions: Chiralpack AD column (Daicel Chemical Industries); Low rate 1 ml/min; Run time 30 min. at 25° C.; Isocratic 15% EtOH (containing 0.1% v/v diethylamine) 85% hexanes (containing 0.1% v/v diethylamine); Retention time of molecule=20 min.

The following abbreviations are employed herein: ACN: acetonitrile; aq.: aqueous; n-BuLi: n-butyllithium; CH₂Cl₂: dichloromethane; CH₃CN: acetonitrile; EtOH: ethanol; EtOAc: ethyl acetate; Eq: equivalents; h: hour(s); HCl: hydrochloric acid; HPLC: high performance liquid chromatography; MeOH: methanol; MgSO₄: magnesium sulfate; min: minutes; MS: mass spectrum; NaHCO₃: sodium bicarbonate; NaOH: sodium hydroxide; Na₂SO₄: sodium sulfate; NH₄Cl: ammonium chloride; Na₂SO₄: sodium sulfate; NMR: nuclear magnetic resonance; RT: room temperature; sat.: saturated; and THF: tetrahydrofuran.

### INTERMEDIATE 1

### 4-Iodo-N,N-diethylbenzamide

To a mixture of 4-iodo-benzoyl chloride (75 g) in 500 mL CH₂Cl₂ was added a mixture of Et₃N (50 mL) and Et₂NH (100 mL) at 0° C. After the addition, the resulting reaction mixture was warmed to RT in 1 h and was then washed with sat. NH₄Cl. The organic extract was dried (Na₂SO₄), filtered and concentrated. Residue was recrystallized from hot hexanes to give 80 g of INTERMEDIATE 1.

### INTERMEDIATE 2

### 4-[hydroxy(3-nitrophenyl)methyl]-N,N-diethylbenzamide

N,N-Diethyl-4-iodobenzamide (5.0 g, 16 mmol) was dissolved in THF (150 mL) and cooled to −78° C. under nitrogen atmosphere. n-BuLi (15 mL, 1.07 M solution in hexane, 16 mmol) was added dropwise during 10 min at −65 to −78° C. The solution was then cannulated into 3-nitrobenzaldehyde (2.4 g, 16 mmol) in toluene/THF (approx. 1:1, 100 mL) at −78° C. NH₄Cl (aq.) was added after 30 min. After concentration in vacuo, extraction with EtOAc/water, drying (MgSO₄) and evaporation of the organic phase, the residue was purified by chromatography on silica (0-75% EtOAc/heptane) to give INTERMEDIATE 2 (2.6 g, 50%). ¹H NMR (400 MHz, CDCl₃) δ_{H} 1.0-1.3 (m, 6H), 3.2 (m, 2H), 3.5 (m, 2H), 5.90 (s, 1H), 7.30-7.40 (m, 4H), 7.50 (m, 1H), 7.70 (d, J=8 Hz, 1H), 8.12 (m, 1H), 8.28 (m, 1H).

### INTERMEDIATE 3

### N,N-diethyl-4-[(3-nitrophenyl)(1-piperazinyl)methyl]benzamide

To a solution of alcohol INTERMEDIATE 2 (10.01 g, 30.5 mmol) in CH₂Cl₂ (200 mL) was added thionyl bromide (2.58 mL, 33.6 mmol). After 1 h at RT the reaction was washed with sat. aq. NaHCO₃ (100 mL) and the organic layer was separated. The aq. layer was washed with CH₂Cl₂ (3×100 mL) and the combined organic extracts were dried (Na₂SO₄), filtered and concentrated. The crude benzyl bromide was dissolved in CH₃CN (350 mL) and piperazine (10.5 g, 122 mmol) was added. After heating the reaction for 1 h at 65° C. the reaction was washed with sat. NH₄Cl/EtOAc and the organic layer was separated. The aq. layer was extracted with EtOAc (3×100 mL) and the combined organic extracts were dried (Na₂SO₄), filtered and concentrated to give racemic INTERMEDIATE 3.

### INTERMEDIATE 4b

### N,N-diethyl-4-[(R)-(3-nitrophenyl)(1-piperazinyl)methyl]benzamide

Racemic INTERMEDIATE 3 was dissolved in EtOH (150 mL) and di-p-toluoyl-D-tartaric acid (11.79 g, 1 Eq.) was added. The product precipitated out over a 12 h period. The solid was collected by filtration and was redissolved in refluxing EtOH until all of the solid dissolved (approximately 1200 mL EtOH). Upon cooling the solid was collected by filtration and the recrystallation repeated a second time. The solid was collected by filtration and was treated with aq. NaOH (2 M) and was extracted with EtOAc. The organic extract was then dried (Na₂SO₄), filtered and concentrated to give 1.986 g of INTERMEDIATE 4b. ¹H NMR (400 MHz, CDCl₃) δ_{H} 1.11 (br s, 3H), 1.25 (br s, 3H), 2.37 (br s, 4H), 2.91 (t, J=5 Hz, 4H), 3.23 (br s, 2H), 3.52 (br s, 2H), 4.38 (s, 1H), 7.31-7.33 (m, 2H), 7.41-7.43 (m, 2H), 7.47 (t, J=8 Hz, 1H), 7.75-7.79 (m, 1H), 8.06-8.09 (m, 1H), 8.30-8.32 (m, 1H).

### INTERMEDIATE 4a

### N,N-diethyl-4-[(S)-(3-nitrophenyl)(1-piperazinyl)methyl]benzamide

INTERMEDIATE 4a may be obtained by performing the following resolution procedure with di-p-toluoyl-L-tartaric acid: Chiralpack AD column (Daicel Chemical Industries); Low rate 1 ml/min.; Run time 30 min. at 25° C.; Isocratic 15% EtOH (containing 0.1% v/v diethylamine) 85% hexanes (containing 0.1% v/v diethylamine); Retention time of molecule=20 min.

### Example 1

### 4-{(S)-(3-aminophenyl)[4-(4-fluorobenzyl)piperazin-1-yl]methyl}-N,N-diethylbenzamide

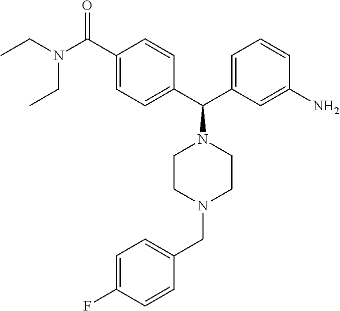

To a solution of INTERMEDIATE 4a (467 mg) in 1,2-dichloroethane (13 ml) was added 4-fluorobenzaldehyde (252 μL; 2 eq) and sodium triacetoxyborohydride (498 mg; 2 Eq.). The reaction was stirred at RT under nitrogen for 18 h and concentrated. Sat. NaHCO₃ was added and the aq. solution was extracted with 3 portions of CH₂Cl₂ and the combined organics were dried over anhydrous Na₂SO₄, filtered and concentrated. The resulting compound was dissolved in a mixture of EtOH, THF, water and sat. NH₄Cl (4 ml; ratios 4:2:1:1 v/v). Iron nanoparticules (3 tips of spatula) were added and the solution was heated at 150° C. for 10 min. in the microwave. The resulting mixture was cooled, filtered through celite and concentrated. The residue was purified by flash chromatography on silica gel, eluting with a gradient from 1%-5% MeOH in CH₂Cl₂. The product obtained was dissolved in CH₂Cl₂ in which 1.2 mL of 1M HCl in ether was added. Solvent was removed and the product was isolated as the hydrochloride salt to give 164 mg Example 1 (30% yield) as a colourless solid. Purity (HPLC): >99%; Optical purity (Chiral HPLC): >99%; ¹H NMR (400 MHz, CD₃OD), 1.08 (t, J=6.5 Hz, 3H), 1.21 (t, J=6.5 Hz, 3H), 3.20-3.26 (m, 4H), 3.51-3.54 (m, 6H), 4.43 (s, 2H), 7.19-7.23 (m, 2H), 7.34 (d, J=8.0 Hz, 1H), 7.40 (d, J=8.0 Hz, 2H), 7.54-7.63 (m, 3H), 7.70-7.82 (m, 4H). Found: C, 54.63; H, 6.49; N, 8.68. C₂₉H₃₆N₄OF×4.1HCl×0.8 H₂O×0.1 C₄H₁₀O has C, 57.67; H, 6.51; N, 8.67%.

### Example 2

### 4-{(R)-(3-aminophenyl)[4-(4-fluorobenzyl)piperazin-1-yl]methyl}-N,N-diethylbenzamide

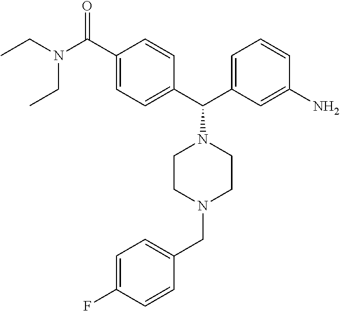

To a solution of INTERMEDIATE 4b (5.790 g, 14.6 mmol) in 1,2-dichloroethane (60 mL) was added 4-fluorobenzaldehyde (2.04 mL, 19.0 mmol) and sodium triacetoxy borohydride (4.02 g, 19.0 mmol). After 20 h at RT the reaction was quenched with aq. NaHCO₃ and the organic layer was separated. The aq. layer was extracted with CH₂Cl₂ (3×100 mL) and the combined organic extracts were dried (Na₂SO₄), filtered and concentrated. The residue was purified by flash chromatography, eluting 30%-50% acetone in hexanes to afford a colourless foam (5.285 g, 71%), which is the nitro intermediate. The nitro intermediate (5.285 g, 10.4 mmol) was dissolved in a mixture of EtOH, THF, water and aq. sat. NH₄Cl (4:2:1:1 ratio v/v) (100 mL) and granules of iron (0.63 mg, 11.5 mmol) were added. The reaction was heated to reflux and periodically more iron granules were added. After 24 h at reflux (90° C.) the reaction was cooled to RT and filtered through celite and concentrated. To the residue was added aq. NaHCO₃ and CH₂Cl₂. The organic layer was separated and the aq. layer was extracted with CH₂Cl₂ (3×100 mL) and the combined organic extracts were dried (Na₂SO₄), filtered and concentrated. The product was purified on silica gel, eluting 1%-5% MeOH in CH₂Cl₂ to afford 3.505 g Example 2 as a pale yellow foam. Impure material was additionally obtained from the above flash chromatography. The impure material was repurified by a second flash chromatography, eluting 100% EtOAc to 5% MeOH in EtOAc to yield a further 0.949 g of Example 2. Combined material obtained: 4.454 g (90% yield). Purity (HPLC): >99%; Optical purity (Chiral HPLC): >99%; ¹H NMR (400 MHz, CD₃OD), 1.08 (t, J=6.5 Hz, 3H), 1.21 (t, J=6.5 Hz, 3H), 3.20-3.26 (m, 4H), 3.51-3.54 (m, 6H), 4.43 (s, 2H), 7.19-7.23 (m, 2H), 7.34 (d, J=8.0 Hz, 1H), 7.40 (d, J=8.0 Hz, 2H), 7.54-7.63 (m, 3H), 7.70-7.82 (m, 4H). Found: C, 54.00; H, 6.34; N, 8.47. C₂₉H₃₅FN4O×4.7HCl×0.2 C₄H₁₀O×0.1 H₂O has C, 54.02; H, 6.37; N, 8.46%.

### Example 3

### 4-{(R)-(3-aminophenyl)[4-(4-fluorobenzyl)piperazin-1-yl]methyl}-N,N-diethylbenzamide sesquifumarate

To a solution of fumaric acid (36.9 g, 318 mmol) in EtOH (700 mL) at 35° C. was added a solution of EXAMPLE 2 (92.3 g, 194 mmol) in EtOH (500 mL), in a controlled manner. The mixing of the two solutions led to crystallisation. The slurry was cooled to 20° C. and stirred at this temperature for a further 16 h. The product was collected by filtration and washed with EtOH (100 mL), before being dried under reduced pressure at 60° C. to provide 114.5 g EXAMPLE 3. Purity (HPLC): >98%; Optical Purity (chiral HPLC): >98%; ¹H NMR (d⁶-DMSO) δ_{H} 1.06 (br, 6H), 2.30 (br, 2H), 2.36 (br, 2H), 2.47 (br, 4H), 3.16 (br, 2H), 3.37 (br, 2H), 3.54 (s, 2H), 4.11 (1H, s), 6.37 (dd, J=1.3 and 7.9 Hz, 1H), 6.55 (d, J=7.6 Hz, 1H), 6.62 (t, J=1.8 Hz, 1H), 6.62 (s, 2H), 6.91 (t, J=7.7 Hz, 1H), 7.13 (t, J=8.8 Hz, J_{HF}=8.8 Hz, 2H), 7.25 (d, J=8.1 Hz, 2H), 7.32 (dd, J=8.4 Hz, J_{HF}=5.8 Hz, 2H), 7.42 (d, J 8.1 Hz, 2H). IR (cm⁻¹) 2973 (NH), 1725 (C═O), 1708 (C═C), 1613 (C═O), 1563 (C═C). Found: C, 62.15; H, 6.35; N, 8.20. 2×C₂₉H₃₅FN₄O with 3×C₄H₄O₄ and 4.5% H₂O requires C, 61.88; H, 6.58; and N, 8.25%.

### Example 4

### Anxiolytic Effect of 4-{(R)-(3-aminophenyl)[4-(4-fluorobenzyl)piperazin-1-yl]methyl}-N,N-diethylbenzamide in the Punished Responding Procedure (a modified Geller-Seifter) in the Rat In Vivo

### Objective

The purpose of the present study was to determine the anxiolytic efficacy of 4-{(R)-(3-aminophenyl)[4-(4-fluorobenzyl)piperazin-1-yl]methyl}-N,N-diethylbenzamide (hereinafter “Compound A”) in the modified Geller-Seifter conflict test.

### Method

In the conflict test, hungry animals were trained to lever-press for food delivery in a standard operant chamber under 2 conditions. In the first condition, referred to as the unsuppressed component, food was delivered on average after 17 lever-presses were made (also called a VR17 schedule of reinforcement). In the second condition, referred to as the suppressed component and signaled by flashing lights inside the operant chamber, food was also delivered following an average of 17 lever-presses, but electric shock was additionally delivered to the floor of the cage under a separate VR17 schedule. Daily sessions consisted of 5 alternating presentations of each component type: suppressed (3 minutes in duration) and unsuppressed (2 minutes in duration). The number of lever presses emitted in the suppressed component was obviously low relative to the unsuppressed component. The rats were dosed with Compound A (0.21 to 21 μmol/kg) and the rate of responding in unpunished and punished components recorded.

### Drug Administration and Preparation

Compound A was dissolved in distilled deionized water/lactic acid and was administered by mouth in a volume of 1 mL/kg body weight. Compound A had a 60 minute pretreatment time. Compound A was administered on Tuesday and Friday and vehicle on Thursday. Monday and Wednesday were washout/baseline days.

### Apparatus

Standard 2-lever operant chambers were used (Med Associates). The chambers were fitted with 2 retractable response levers and a stimulus lamp over each of the 2 levers. A pellet dispenser delivered 45 mg food pellets, (Bio Serv) to a cup located inside of the chamber below and between the 2 response levers. A lamp at the top and back of the chamber served as houselights. The grid floors of the operant chambers were interfaced to shock generators and scramblers (Med Associates). All events in the chambers were controlled and monitored by a microprocessor.

### Procedures

There were 2 components in the procedure: 1) an unsuppressed responding component (unpunished) with 2 minutes in duration; and 2) a suppressed responding component (punished) with 3 minutes in duration.

In the unpunished component, the houselights and both stimulus lamps over the response levers were turned on, the lever on the left-hand side of the chamber extended, and a food pellet was delivered following an average of 17 responses on the lever in the chamber (range 3 to 40 responses)—a variable-ratio 17 schedule (VR17).

In the punished component (which followed the unpunished component), the right-hand lever was extended into the chamber; the stimulus lamps and houselights were turned on and off at 1 second intervals, in succession, which served as a cue for this component; and food was available under a VR17 schedule, but was accompanied by an electrical current (0.5 second duration) that was delivered to the grid floor of the chamber under an independent VR17 schedule. The level of the current was adjusted for each individual subject until responding was reduced in the punished component to a level that was about 5% to 10% that of the unpunished component, and ranged from 0.2 mA to 0.75 mA. Unpunished and punished components were separated by 10 second time-out periods in which both response levers were retracted and all stimulus lamps turned off 2 minute unpunished and 3 minute punished components alternated until 5 of each were completed. Daily sessions always began with the unpunished responding component. For any given drug test, rats whose responding was most stable were chosen from a larger pool of trained rats. Several doses were tested on a given test day in different subjects. Each dose, then, was tested in different sub-set of rats.

### Data Analysis

The dependent variables recorded were the rate of responding in unpunished and punished components (total responses/total time under the component) and the number of shocks delivered. A selective anxiolytic effect is defined as an increase in responding in the punished components with relatively less or no effect on responding in unpunished components. t-Tests were used to compare mean of the control's rate of responding on vehicle day of the rats used for a specific dose to the same rats means following delivery of each dose of Compound A (for only the rats used within each dose).

### Results

The results of the present study are summarized in Tables 2 and 3 set forth hereinbelow. Compound A increased the rate of punished responding at 0.63, 2.11, 6.3, and 21.1 μmol/kg compared to their vehicle controls. Compound A was maximally efficacious at 2.11 μmol/kg, producing about 300% increase in punished responding. 4 Compound A at 0.21 μmol/kg was not effective in this model. Compound A did not significantly decrease unpunished responding at the doses tested.

TABLE 2
Rate of responding test result summary

| | Punished | | | Unpunished | | |
|---|---|---|---|---|---|---|
| Treatment | responding | Vehicle | P value | responding | Vehicle | P value |
| Vehicle (average) | 0.10 ± 0.01 | ND | ND | 1.92 ± 0.05 | ND | ND |
| Compound A | 0.11 ± 0.02 | 0.11 ± 0.01 | 0.493 | 2.06 ± 0.11 | 2.01 ± 0.11 | 0.080 |
| 0.21 μmol/kg | | | | | | |
| Compound A | 0.24 ± 0.03 | 0.09 ± 0.01 | 0.003^{a} | 2.02 ± 0.14 | 2.19 ± 0.06 | 0.092 |
| 0.63 μmol/kg | | | | | | |
| Compound A | 0.18 ± 0.04 | 0.08 ± 0.02 | 0.046^{a} | 1.95 ± 0.15 | 1.87 ± 0.17 | 0.241 |
| 2.1 μmol/kg | | | | | | |
| Compound A | 0.17 ± 0.03 | 0.09 ± 0.01 | 0.031^{a} | 1.92 ± 0.13 | 1.81 ± 0.11 | 0.031^{a} |
| 6.3 μmol/kg | | | | | | |
| Compound A | 0.20 ± 0.04 | 0.10 ± 0.021 | 0.045^{a} | 1.69 ± 0.12 | 1.77 ± 0.09 | 0.169 |
| 21.1 μmol/kg | | | | | | |

^{a}P value is associated with t-Test vs individual vehicle group (p < 0.05)
ND = Not determined

TABLE 3
Percent of control test result summary

| | Punished | | | Unpunished | | |
|---|---|---|---|---|---|---|
| Treatment | responding | Vehicle | P value | responding | Vehicle | P value |
| Vehicle. (average) | 95.5 ± 6.61 | ND | ND | 100 ± 2.85 | ND | ND |
| Compound A | 107 ± 19.3 | 110 ± 14.9 | 0.458 | 103 ± 1.63 | 105 ± 5.76 | 0.396 |
| 0.21 μmol/kg | | | | | | |
| Compound A | 273 ± 39.2 | 92.7 ± 8.70 | 0.005^{a} | 92.1 ± 5.12 | 114 ± 3.30 | 0.003^{a} |
| 0.63 μmol/kg | | | | | | |
| Compound A | 298 ± 132 | 80.4 ± 16.0 | 0.092 | 106 ± 5.60 | 97.3 ± 9.10 | 0.241 |
| 2.1 μmol/kg | | | | | | |
| Compound A | 207 ± 67.2 | 91.8 ± 11.7 | 0.079 | 106 ± 2.94 | 94.3 ± 5.80 | 0.031^{a} |
| 6.3 μmol/kg | | | | | | |
| Compound A | 272 ± 100 | 104 ± 24.0 | 0.107 | 95.5 ± 4.08 | 92.3 ± 4.64 | 0.317 |
| 21.1 μmol/kg | | | | | | |

^{a}P value is associated with t-Test vs individual vehicle group (p < 0.05)
ND = Not determined

### Conclusion

Compound A has acute anxiolytic activity in a modified Geller-Seifter conflict model with a level of efficacy to that of diazepam (˜250% at 3.5 μmol/kg by mouth; data not shown). Observations of test subjects in their home cage suggest Compound A does not possess sedative/hyperactive properties.

### Example 5

### Antidepressant effect of 4-{(R)-(3-aminophenyl)[4-(4-fluorobenzyl)piperazin-1-yl]methyl}-N,N-diethylbenzamide in the learned helplessness procedure in the rat

### Objective

The purpose of the present study was to determine the antidepressant potential of 4-{(R)-(3-aminophenyl)[4-(4-fluorobenzyl)piperazin-1-yl]methyl}-N,N-diethylbenzamide (hereinafter “Compound A”) in the learned helplessness test.

### Method

Male Sprague Dawley rats were exposed to inescapable electrical stimulation for 2 successive, daily 1 hour periods (conditioning), and then subsequently trained to avoid or escape the electrical stimulation by running to the opposite side of the test cage. During conditioning and training, animals were injected twice per day with vehicle, imipramine HCl (15 mg/kg) or 0.1, 1 and 10 mg/kg Compound A over 2 separate studies. In the training phase, when the electrical stimulation was escapable, the number of escape failures was recorded. Standard shuttle cages (20 L×16 W×21 centimeters H) fitted with a grid floor were used. The chambers could be partitioned with a closed partition or with an archway that allowed the animals to pass between the 2 sides of the cage. A computer controlled and monitored all events in the chamber.

### Procedure

The procedure utilized involved an induction phase and an avoidance training phase. In the induction phase, rats were enclosed in one side of the shuttle cage and electrical stimulation (2 mA, 9.9 second duration) was delivered to the floor of the cage every 2, 5 or 10 seconds (randomly selected for each trial) until 90 electrical stimulations were delivered. Subjects had no opportunity to escape or to avoid the electrical stimulation. Induction was conducted for 2 consecutive days. In the avoidance training phase, a partition with an arch through which the rats could pass was inserted into the center of the shuttle cages. The method employed a standard shuttle avoidance in which a compound, conditioned stimulus (a 5 second presentation of a tone and turning on of a lamp on the side of the rat containing cage) served to indicate impending presentation of electrical stimulation to the floor of the cage. The electrical stimulation was presented for a 5 second period 5 seconds after initiation of the conditioned stimulus. Entry into the opposite side of the shuttle cage via the arched partition prior to onset of electrical stimulation resulted in the end of the trial (avoidance response). If electrical stimulation was delivered, entry into the opposite side of the cage resulted in termination of the electrical stimulation and the Conditioned Stimulus (escape). A 30 second inter-trial interval was employed. 40 minute avoidance training sessions, consisting of 50 trials were conducted on 2 consecutive days, beginning 48 hours after the final induction session.

### Drug Administration and Preparation

Dosages of all compounds are reported as the free base. Imipramine and Compound A were dissolved in distilled deionized water and administered by mouth in a volume of 1 mL/kg body weight. Drug was administered immediately following conditioning and training sessions and approximately 7-8 hrs after the first injection, as well as on the day in the middle of the study when no conditioning or training was conducted. In study 2, imipramine was administered 30 minutes prior to avoidance training whereas in all other circumstances, drug was administered following training.

### Data Analysis

The primary dependent variable was escape failures during avoidance training. Additionally, because some delta opioid agonists have been shown to produce locomotor stimulation, center crossings during avoidance training were also recorded and compared among groups, which allows a gauge of motor activity. An increase in center crossings with respect to vehicle control suggests that locomotor stimulation may be partly or fully responsible for the putative antidepressant effects of the compound. T-Tests were used to compare the performance of the vehicle-administered group to drug treated groups. The no-induction group was used to gauge whether learned helplessness was established, by comparison to the vehicle treated group.

### Results

The results of the present study are summarized in Table 4 set forth hereinbelow. Saline-treated rats exposed to inescapable electric stimulation (induction phase) failed to escape in the avoidance phase in 16 (on average) of the 50 trials. The study integrity was confirmed by a significantly reduced number of escape failures in saline-treated rats that were not exposed to inescapable electric stimulation and the imipramine-treated rats. All 3 doses of Compound A tended to decrease escape failures, although only the 2 higher doses were significantly different from the saline-treated rats exposed to inescapable electric stimulation. The reduction in escape failures was not due to increased locomotion per se, since center crossings in the cage in the treated groups was either not different or even slightly reduced compared to the saline-treated rats exposed to inescapable electric stimulation.

TABLE 4

| | Mean Escape | P | Mean Center | P |
|---|---|---|---|---|
| Treatments | Failures ± (SEM) | Value | Crossings ± SEM | Value |
| IES + Saline | 16.2 (3.9) | — | — | — |
| No IES + | 5.4 (1.4) | <0.003* | 32.9 (2.1) | >.05 |
| Saline | | | | |
| Imipramine | 2.3 (1) | <0.002* | 30.2 (1.1) | <0.02* |
| 20 mg/kg | | | | |
| Compound A | 6.1 (3.4) | =0.06 | 26.6 (0.85) | >0.05 |
| 0.1 mg/kg | | | | |
| Compound A | 3.1 (1.7) | <0.004* | 30.9 (2.9) | >0.05 |
| 1 mg/kg | | | | |
| Compound A | 4.9 (2) | <0.01* | 26.9 (0.7) | <0.02* |
| 10 mg/kg | | | | |

IES = inescapable electrical stimulation.
P value is associated with T test vs. IES + vehicle group.
All treatments were given by mouth twice a day.

### Conclusion

Compound A produced a decrease in escape failures in the learned helplessness test, which is indicative of potential antidepressant action.

### Example 6

### Treatment of AMDD patients with 4-{(R)-(3-aminophenyl)[4-(4-fluorobenzyl)piperazin-1-yl]methyl}-N,N-diethylbenzamide sesquifumarate

### Overview

A randomized, double blind, placebo-controlled, parallel-group experimental study was designed to assess the efficacy and safety of 3 mg of 4-{(R)-(3-aminophenyl)[4-(4-fluorobenzyl)piperazin-1-yl]methyl}-N,N-diethylbenzamide sesquifumarate (hereinafter “compound B”) given twice daily over 4 weeks to male and female patients ages 18 to 65 years meeting diagnostic criteria for AMDD but without psychotic features, according to a Hamilton Rating Scale for Depression (hereinafter “HRSD17”) and Hamilton Psychiatric Rating Scale for Anxiety (hereinafter “HAM-A”) total scores and DSM-IV and confirmed by the Structured Clinical Interview for the Diagnostic Manual of Mental Disorders, Fourth Edition, Patient Version (hereinafter “SCID-I/P”) (See, First M B, Spitzer R L, Gibbon M, Williams A R (2001): Structured Clinical Interview for DSM-IV TR Axis I Disorders, Research Version, Patient Edition. New York: New York State Psychiatric Institute, Biometrics Research).

More specifically, patients involved in this study will have 1) documented clinical diagnosis meeting criteria from the DSM-IV for at least one of the following: 296.22 Major Depressive Disorder, Single Episode, Moderate; 296.23 Major Depressive Disorder, Single Episode, Severe Without Psychotic Features, duration at least 1 year; 296.32, Major Depressive Disorder, Recurrent, Moderate; and/or 296.33, Major Depressive Disorder, Recurrent, Severe Without Psychotic Features; and 2) a HRSD17 total score ≧20; a HAM-A total score ≧16; and a Clinical Global Impressions Severity (hereinafter “CGI-S”) score ≧4 at both enrollment and randomization.

The HRSD17 is a widely used observational rating measure of depression severity. (See, Hamilton M (1960b): A rating scale for depression. J Neurol Neurosurg Psychiatry 23:56-62). The 17-item version of this scale, which also referred to as HAMD, will be administered to assess the severity of depression. The HAMD assesses both the presence and severity of individual signs and symptoms characterizing depression without psychotic features.

HAM-A is a widely used observational rating measure of anxiety severity. The scale consists of 14 items. Each item is rated on a scale of 0 to 4. This scale will be administered to assess the severity of anxiety and its improvement during the course of therapy. The HAM-A total score is the sum of the 14 items and the score ranges from 0 to 56.

The Hamilton Rating Scale for Depression Anxiety/Somatization Subscale (hereinafter “HRSD17(A/S)”) is a subscale of the HRSD17. It factors scores derived from items 10, 11, 12, 13, 15, and 17 of the HRSD17.

The SCID I/P is a semi-structured interview. It is administered by a clinician to diagnose psychiatric illness and provides probe questions as well as follow-up questions to assist the clinician in diagnosis. It includes an overview to obtain information about demographics, work, chief complaint, history of present illness, past history, treatment history, and current functioning. The main body of the SCID I/P includes 9 modules designed to diagnose 51 mental illnesses in all. The modules of the research version can be tailored to needs, purpose, and goals of the investigation. It includes sections on current as well as past psychiatric disorders.

CGI-I/S scale is a three-item scale that assesses treatment response in psychiatric patients. (See, Guy W (1976): ECDEU Assessment Manual for Psychopharmacology, Revised). The administration time is 5 minutes. The scale consists of three items: Severity of Illness (item 1); Global Improvement (item 2); and Efficacy Index (item 3). Item 1 is rated on a seven-point scale from 1, which is normal to 7, which is among the most extremely ill patients. Item 2 is also rated on a seven-point scale from 1, which is very much improved to 7, which is very much worse. Each includes an additional response of “not assessed”. Item 3 is rated on a four-point scale from “none” to “outweighs therapeutic effect”. Items 1 and 3 are assessed based on the previous week's experience. Item 2 is assessed from the period since the initiation of the current treatment.

CGI-S scale is the Item 1 Severity of Illness scale of the CGI-I/S scale.

Approximately 96 subjects with AMDD will be screened to randomize 80 in this study. Randomization will be performed at a 2:1 ratio into the following two groups:

Treatment Group A: On the morning of Day 1, all patients in treatment group A will receive a 3 mg dose of compound B. On Day 2, the dose will be increased 3 mg of compound B twice a day. Patients will continue on 3 mg of compound B twice a day for approximately 28 days; and

Treatment Group B: Patients in treatment Group B will receive placebo capsules matching the color, size and appearance of the capsules received by treatment group A. The regimen for dosing will be identical to Treatment Group A's dosing regimen.

Dose selection: In rodents, doses of Compound B that produced mean plasma exposures ≧2 ng/ml have been shown to be efficacious in separate tests of anxiolysis and antidepressant activity. Monte Carlo simulations (N=1000) based on human pharmacokinetic data for Compound collected in the Phase I program (N=96 male subjects) estimates 3 mg/kg twice a day will achieve an average plasma exposure of ≧2 ng/ml in 96% of subjects upon initial dosing, increasing to >98% of subjects at steady state.

### Screening Period

The screening period will be up to 30 days prior to Day 1 of the Treatment Period. All patients will be required to stop current antidepressant treatment at least 14 days prior to Day 1 of the Treatment Period. Patients may be admitted to the Clinical Research Center (hereinafter “CRC”) during the washout period based on deterioration of their depressive symptoms.

### Treatment Period

Day 1 to Day 7: On Day 1 of the Treatment Period, patients will be randomized to receive either compound B (Treatment Group A) or placebo (Treatment Group B) based on the randomization schedule.

Day 7 to Day 28: Patients will return to the CRC for 3 scheduled visits at Weeks 2, 3 and 4 during the Treatment Period for safety, tolerability, and efficacy assessments.

### Follow-Up Visit

Patients will be asked to return for a follow-up visit 7-10 days after completion of the outpatient period. During the follow-up visit, additional safety, tolerability, and efficacy assessments will be administered.

### Data Analysis

This study is designed to assess the efficacy of 28 days of 3 mg of Compound B by mouth twice a day compared with placebo in improving overall depressive symptomatology in patients with AMDD. The primary efficacy analysis will compare the response rates of Compound B versus placebo groups in the intent-to-treat sample at study endpoint. Response will be defined as a reduction of ≧50% in the HRSD17 or HAM-A total score, and a CGI-I score=1 or 2 at Week 4 of treatment or study endpoint if the study is not completed. Additionally, the following analyses of HRSD17 and HAM-A data will be conducted:
Change from baseline to day of discharge from the CRC and change from baseline to Weeks 1, 2, 3, and 4 of treatment and change from baseline to follow-up in HRSD17 and HAM-A total scores.
Change from baseline to day of discharge from the CRC and change from baseline to Weeks 1, 2, 3, and 4 of treatment and change from baseline to follow-up in HRSD17 (A/S), and HRSD17, Item 10 (Anxiety Psychic) scores.
Time to anxiolytic response: defined as first assessment of a ≧50% reduction in HAM-A or HRSD17 (A/S) score using Kaplan-Meier survival analysis method.
Time to antidepressant response: defined as first assessment of a ≧50% reduction in HRSD17 total score using Kaplan-Meier survival analysis method.

All statistical comparisons will be based on a 2-tailed test using an alpha level of 0.05 unless otherwise specified. No correction to the reported p-values will be made for the primary analysis.

Descriptive statistics for continuous data will include number (n), mean, median, standard deviation, minimum and maximum value. Descriptive data for categorical data will include n, frequency, and percentage.

Efficacy analyses will be performed in the intent-to-treat population, and safety analyses will be performed in the safety population unless otherwise specified.

Descriptive statistics will be provided for all efficacy variables. Additionally, the ANCOVA model will be used for continuous variable and the logistic regression model will be used for categorical variables. For the time to event variables, Cox proportional hazards model will be used.

Clinical assessments including HRSD17, HAM-A, and CGI-I/S will be conducted. The HRSD17 will be used to collect information on depressive symptoms; the HAM-A will be used to collect data on anxiety symptoms; and the CGI-I/S will be used to collect data on overall severity of improvement/illness. In addition, information on depression, anxiety, pain symptoms, and suicidal ideation will be collected using the Inventory of Depressive Symptomatology, Clinician and Subject Rated (hereinafter “IDS-C₃₀/IDS-S₃₀”), the Depression Anxiety Stress Scale (hereinafter “DASS₄₂”), the 23-item Kellner Somatic Symptom Questionnaire (hereinafter “SSQ”), and a pain scale. The proportion who show a clinically significant change from baseline to endpoint in the HRSD17 or the HAM-A total scores (Hamilton 1960a; Hamilton 1967) as well as the CGI-I/S will serve as the primary efficacy measure for the study. Secondary efficacy assessments include the HRSD17, HAM-A, CGI-I/S, Inventory of Depressive Symptomatology, Clinician and Subject Rated (hereinafter “IDS-C₃₀/IDS-S₃₀”), Depression Anxiety Stress Scale (hereinafter “DASS₄₂”), and the pain assessment. In addition to indicating simple change in severity, the HRSD17 total score will be used to dichotomize patients into response versus nonresponse categories at the end of the Study. A responder will be defined as any patient who demonstrates a 50% or greater decrease in HRSD17 total score from baseline to endpoint. A remitter will be defined as any patient who demonstrates a <7 HRSD17 total score.

The IDS-C₃₀/IDS-S₃₀ is a 30-item observational rating measure of depression severity. The estimated time to administer this scale is 10 minutes. The scores on this measure can range from 0 to 84.

The DASS₄₂ is a set of 3 self-report scales measuring the negative emotional states of depression, anxiety and stress. This 42-item questionnaire has been shown to have high internal consistency and to yield meaningful discriminations in a variety of settings. The depression scale assesses dysphoria, hopelessness, devaluation of life, self-deprecation, lack of interest/involvement, anhedonia, and inertia. The anxiety scale assesses autonomic arousal, skeletal muscle effects, situational anxiety, and subjective experience of anxious affect. The stress scale is sensitive to levels of chronic non-specific arousal. It assesses difficulty relaxing, nervous arousal, and being easily upset/agitated, irritable/over-reactive and impatient.

The SSQ is a 23-item somatic scale composed of items that include both negative (17 items) and positive (6 items) somatic symptoms. The 17 negative somatic symptoms are as follows: feeling of not having enough air, heavy arms or legs, appetite poor, tight head and neck, choking feeling, feeling of pressure in head or body, weak arms or legs, breathing difficult, parts of the body feel numb or tingling, heart beating fast or pounding, pressure on head, nauseated/sick to stomach, upset bowels or stomach, muscles pains, headaches, cramps, and head pains. The six positive somatic symptoms are as follows: feeling healthy, feeling fit, no pains anywhere, arms and legs feel strong, no aches anywhere, and no unpleasant feelings in head or body.

Exemplary pain scales include but are not limited to, for example, the VAS and Likert Pain Scales. The VAS pain scale is a visual analog scale that assists patients in subjectively rating pain or sequelae of pain. The VAS is a straight line (100 mm) with the left end of the line representing no pain or symptoms and the right end of the line representing the worst pain or related symptoms imaginable. Patients rate their pain/symptoms by marking on the line where they feel their pain/symptoms lie. Likert scales are numbered scales to indicate level of pain.

1-24. (canceled)

25. A method for treating anxious major depressive disorder in a warm-blooded animal, comprising:
administering to a subject afflicted with anxious major depressive disorder in need of such treatment a therapeutically effective amount of a compound 4-{(3-aminophenyl)[4-(4-fluorobenzyl)piperazin-1-yl]methyl}-N,N-diethylbenzamide or an enantiomer thereof, or a pharmaceutically acceptable salt thereof, or mixture of said compound and said salt.

26. A method according to claim 25, wherein said compound is 4-{(R)-(3-aminophenyl)[4-(4-fluorobenzyl)piperazin-1-yl]methyl}-N,N-diethylbenzamide, or a pharmaceutically acceptable salt thereof, or mixture of said compound and said salt.

27. A method according to claim 26, wherein said compound comprises 4-{(R)-(3-aminophenyl)[4-(4-fluorobenzyl)piperazin-1-yl]methyl}-N,N-diethylbenzamide sesquifumarate.

28. A method according to claim 26, wherein said compound comprises an enantiomerically pure compound comprising at least 75% 4-{(R)-(3-aminophenyl)[4-(4-fluorobenzyl)piperazin-1-yl]methyl}-N,N-diethylbenzamide, or a pharmaceutically acceptable salt thereof, or mixture of said compound and said salt.

29. A method according to claim 28, wherein the enantiomerically pure compound comprises at least 90% 4-{(R)-(3-aminophenyl)[4-(4-fluorobenzyl)piperazin-1-yl]methyl}-N,N-diethylbenzamide, or a pharmaceutically acceptable salt thereof, or mixture of said compound and said salt.

30. A method according to claim 28, wherein the enantiomerically pure compound comprises at least 95% 4-{(R)-(3-aminophenyl)[4-(4-fluorobenzyl)piperazin-1-yl]methyl}-N,N-diethylbenzamide, or a pharmaceutically acceptable salt thereof, or mixture of said compound and said salt.

31. A method for treating anxious major depressive disorder in a warm-blooded animal, comprising:
administering to a subject afflicted with anxious major depressive disorder in need of such treatment a therapeutically effective amount of a pharmaceutical composition comprising a therapeutically effective amount of a compound comprising 4-{(3-aminophenyl)[4-(4-fluorobenzyl)piperazin-1-yl]methyl}-N,N-diethylbenzamide or an enantiomer thereof, or a pharmaceutically acceptable salt thereof, or a mixture of said compound and said salt, and a pharmaceutically acceptable carrier.

32. A method according to claim 31, wherein said compound is 4-{(R)-(3-aminophenyl)[4-(4-fluorobenzyl)piperazin-1-yl]methyl}-N,N-diethylbenzamide, or a pharmaceutically acceptable salt thereof, or a mixture of said compound and said salt, and a pharmaceutically acceptable carrier.

33. A method according to claim 31, wherein said compound comprises 4-{(R)-(3-aminophenyl)[4-(4-fluorobenzyl)piperazin-1-yl]methyl}-N,N-diethylbenzamide sesquifumarate.

34. A method according to claim 31, wherein said compound comprises an enantiomerically pure compound comprising at least 75% 4-{(R)-(3-aminophenyl)[4-(4-fluorobenzyl)piperazin-1-yl]methyl}-N,N-diethylbenzamide, or a pharmaceutically acceptable salt thereof, or a mixture of said compound and said salt, and a pharmaceutically acceptable carrier.

35. A method according to claim 34, wherein said compound comprises an enantiomerically pure compound comprising at least 90% 4-{(R)-(3-aminophenyl)[4-(4-fluorobenzyl)piperazin-1-yl]methyl}-N,N-diethylbenzamide, or a pharmaceutically acceptable salt thereof, or a mixture of said compound and said salt, and a pharmaceutically acceptable carrier.

36. A method according to claim 34, wherein said compound comprises an enantiomerically pure compound comprising at least 95% 4-{(R)-(3-aminophenyl)[4-(4-fluorobenzyl)piperazin-1-yl]methyl}-N,N-diethylbenzamide, or a pharmaceutically acceptable salt thereof, or a mixture of said compound and said salt, and a pharmaceutically acceptable carrier.

37. A pharmaceutical composition comprising a therapeutically effective amount of a compound 4-{(3-aminophenyl)[4-(4-fluorobenzyl)piperazin-1-yl]methyl}-N,N-diethylbenzamide or an enantiomer thereof, or a pharmaceutically acceptable salt thereof, or a mixture of said compound and said salt, and a pharmaceutically acceptable carrier for the treatment of anxious major depressive disorder.
